(19) United States
(12) Patent Application Publication
(10) Pub. No.: US 2009/0157021 A1
(43) Pub. Date: Jun. 18, 2009

(54) ARTICLES WITH INFORMATIVE PATTERNS, AND METHODS OF MAKING THE SAME

(76) Inventors: Suzanne K. Sullivan, Combined Locks, WI (US); Katie A. Boland, Menasha, WI (US); Garry Roland Woltman, Appleton, WI (US)

Correspondence Address: Christopher M. Goff (27839);ARMSTRONG TEASDALE LLP, ONE METROPOLITAN SQUARE, SUITE 2600, ST. LOUIS, MO, 63102, US

(21) Appl. No.: 11/957,163

(22) Filed: Dec. 14, 2007

(51) Int. Cl. A61F 13/49

(52) U.S. Cl. 604/359; 604/385.01

(57) ABSTRACT

A plurality of graphical elements form optical illusions associated with consumer products to aid in conveying visual perception of desired features. The optical illusions may provide perceptions of motion and/or elevation deviation into or out of a surface. Further, graphical elements sharing a single common color tone may create one or more of the illusions that may otherwise be enhanced by incorporating multiple color tones to the graphical elements creating the illusions. Exemplary consumer products include disposable diapers, feminine sanitary napkins, printed or electronic advertising media, and removable packaging enclosing articles prior to their use.

### BACKGROUND OF THE INVENTION

Various consumer products provide benefits not readily apparent to a user. These benefits may be indiscernibly incorporated internally within the product or may be unperceivable or invisible on a surface of the product. For example, disposable diapers and sanitary napkins utilize an internal absorbent media in which the level of absorbency does not necessarily depend on its relative thickness. Accordingly, consumer product selection based on thickness is misleading in this case.

Without a visual indication relating to features of the products, the users often select a less appropriate product offering than one that would better meet their needs. The user may not perceive added features new to a particular product or technological advancements being utilized.

### SUMMARY OF THE INVENTION

One embodiment relates to an absorbent personal care article. The article includes a body having internal absorbent media and an exterior surface. A plurality of graphical elements visible along the exterior surface forms a pattern that creates an optical illusion of motion.

A personal care article in one embodiment includes a body having a region containing a visually indiscernible component of the article that provides at least one functional aspect associated with use of the article. The article further includes a plurality of graphical elements visible on the body at the region containing the indiscernible component. Due to arrangement of the graphical elements relative to one another, a viewer perceives at least one of the elements with a first characteristic different than a second characteristic, later in time, dependent on a focal point of the viewer even though the graphical elements are static.

For one embodiment, an absorbent personal care article includes a body having internal absorbent media between a first liquid pervious cover layer and a second liquid impervious layer. A plurality of graphical elements appear visible on an exterior surface of the body from a side of the body having the first liquid pervious cover layer. Arrangement of the graphical elements relative to one another forms an optical illusion of motion perceivable as at least one of the elements appearing to move away from an edge of the body.

### BRIEF DESCRIPTION OF THE DRAWINGS

FIG. 1 illustrates an exemplary optical illusion of motion formed by a plurality of graphical elements disposed on a substrate, in accordance with one aspect of the invention.

FIG. 2 illustrates an application in which a feminine hygiene pad utilizes an optical illusion of motion and elevation deviation, in accordance with one aspect of the invention.

FIG. 3 illustrates a schematic cross section view of the pad taken across line 3-3 in FIG. 2.

FIGS. 4-6 illustrate exemplary optical illusions viewable on external surfaces of substrates, in accordance with aspects of the invention.

FIGS. 7 and 8 illustrate additional exemplary feminine hygiene pads utilizing optical illusions in which graphical elements are shaped and arranged to define first and second patterns with the second pattern surrounding the first pattern and different from the first pattern, in accordance with aspects of the invention.

### DETAILED DESCRIPTION

The following description references embodiments of the invention. However, it should be understood that the invention is not limited to any specifically described embodiments. Instead, any combination of the following features and elements, whether related to different embodiments or not, is contemplated to implement and practice the invention. Furthermore, in various embodiments the invention provides numerous advantages over the prior art. However, although embodiments of the invention may achieve advantages over other possible solutions and/or over the prior art, whether or not a particular advantage is achieved by a given embodiment is not limiting of the invention. Thus, the following aspects, features, embodiments and advantages are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

Embodiments of the invention generally relate to optical illusions formed by a plurality of graphical elements associated with articles of manufacture (e.g., consumer products) to aid in conveying visual perception of desired features. The optical illusions may provide perceptions of motion and/or contour (e.g., elevation deviation into or out of a surface). Further, graphical elements sharing a single common color tone may create one or more of the illusions that may otherwise be enhanced by incorporating multiple color tones to the graphical elements creating the illusions. Exemplary consumer products include disposable diapers, feminine sanitary napkins, printed or electronic advertising media, and removable packaging enclosing articles prior to their use. The consumer products may be disposable, meaning that the product rather than being laundered or otherwise restored for use is designed to be discarded after a single or limited use once its ability to perform its intended function is exhausted. In addition to intuitively conveying information about features of the products, perceivable cues provide the user with added confidence in the products, guide correct use of the products, and facilitate marketing of the products, according to various embodiments.

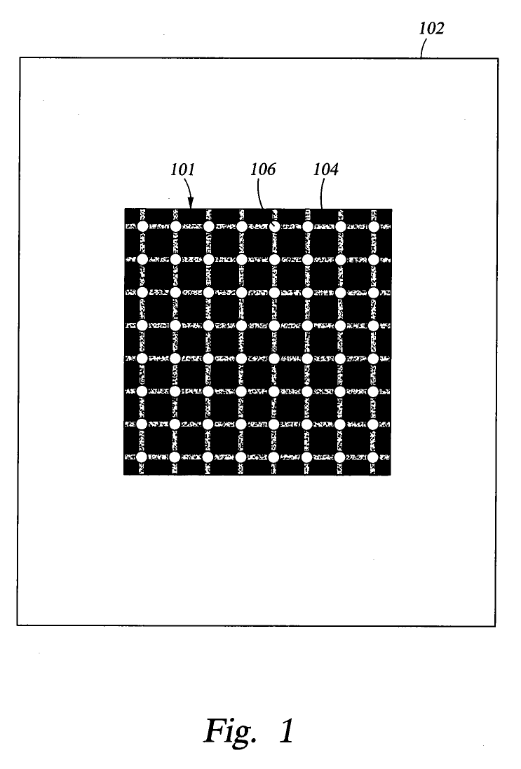

FIG. 1 shows an exemplary optical illusion of motion formed by a plurality of graphical elements 101 disposed on a substrate 102. The graphical elements 101 include black squares 104 formed by a matrix of rows and columns having a relatively lighter shade than the black squares. Illustratively, FIG. 1 shows an 8×8 matrix; however, the size of the matrix may vary and the number of columns may be different from the number of rows. White dots 106 are formed at the intersections of the columns and rows. This arrangement of the graphical elements 101 creates a scintillating grid illusion where black dots appear and disappear within the white dots 106. While all the graphical elements 101 are static, a viewer perceives at least one of the white dots 106 with a white characteristic different than a black characteristic perceived, later in time, as the viewer changes their focal point, thereby making an appearance of change or motion among the dots 106 in the absence of actual physical movement.

The graphical elements 101 thus draw the gaze of a user to a certain area of the substrate 102. By drawing the gaze of the user, the graphical elements 101 may highlight a benefit associated with the substrate 102 and/or guide the user in proper application of the substrate 102. The illusion chosen also enables intuitive highlighting of the benefit provided depending on applications for the substrate 102. Exemplary benefits as discussed further herein include protection, comfort, convenience, discretion, and health. For example, the graphical elements 101 in some embodiments identify, with activity among the dots 106, where on the substrate 102 a medication with active chemical compounds is located or where on the substrate 102 the user is supposed to apply a stimulus (e.g., urine, menses, pressure) to activate or utilize a substance of the substrate 102.

The substrate 102 represents any body such as disposable diapers, feminine sanitary napkins, printed or electronic advertising media (e.g., television and the Internet), and removable packaging enclosing articles prior to their use. For some embodiments, the substrate 102 defines a bandage, a skin wellness product, a medicated pad or wipe, an applicator, a test product (e.g., a disposable pregnancy test), surgical drapes, gowns, sterile wraps, any feminine hygiene products (e.g., the sanitary napkins, liners, tampons, interlabial devices), infant diapers, children's training pants, adult incontinence products, absorbent wipes, and covering mats. Absorbent articles, in some embodiments, form the substrate 102 such that the substrate 102 absorbs and contains body liquids and may be placed against or near the skin to absorb and contain the various liquids discharged from the body.

The packaging that may form the substrate 102 and enclose the articles prior to their use may include individual wrappers each containing a single one of the articles or containers each holding a plurality of the articles with, or without, the individual wrappers. For some embodiments, packaging may be transparent or translucent or include a window such that the graphical elements 101 disposed on the substrate 102 (e.g., a pad) located inside the packaging are visible. The packaging thus can enable conveying the illusion of the graphical elements 101 to the user whether or not the substrate 102 is the packaging itself or the article within the packaging.

In one embodiment, the substrate 102 provides a diaper having a chemical or compound (e.g., activated carbon, silica, cyclodextrin, or a carboxylic acid based odor absorbing compound) that activates with moisture to control odor. The graphical elements 101 correspond to where the activation occurs and signify to the user this additional benefit not otherwise visible. For another exemplary embodiment, graphical elements 101 identify where an invisible therapeutic film is contained on a bandage that is provided by the substrate 102. For some embodiments, text can be included with graphics that create illusions, such as those described herein, and/or a patterned area shaped to define text can incorporate the graphics that create the illusions. For example, block lettering “Rx” may be filled in with the pattern shown in FIG. 1 on the bandage to reinforce presence of medication or a prescription compound.

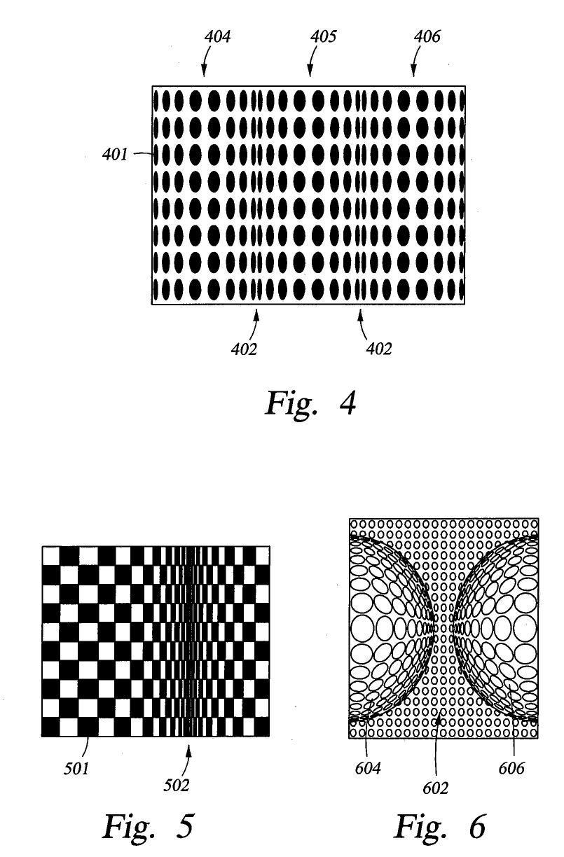

The substrate 102 in one embodiment provides a training pant with the graphical elements 101 visible along an outer cover (analogous to the liquid impervious backsheet 302 shown in FIG. 3) forming an exterior surface opposite a user facing surface of the pant such that the graphical elements 101 appear as moving objects on an outside of the pant. As discussed further herein, the graphical elements 101 may disappear or appear upon the substrate 102 absorbing urine. For some embodiments, optical illusions of motion as demonstrated by example with the graphical elements 101 may be applied to characters, animals or cartoon figures. Regardless of how the illusion of motion is implemented, the illusion of motion highlights enhanced aspects of the pant relating to movement ability by appearing to allow the user to move more freely. The motion provided by the graphical elements 101 therefore correlates to activity of toddlers to indicate that the pants are designed to enable such movement with comfort and ease.

Various optical illusions are contemplated and are suitable for creating the illusions on the substrate 102 regardless of any particular theory or technique employed with respect to arranging any graphically elements in a pattern to create the illusion. The graphical elements may include any figure, open or closed shape, or line such as those shown in the figures herein. The pattern formed by a plurality of these graphical elements creates the optical illusions. By way of example, lines with varying widths and/or varying spacing between adjacent lines provide a selected arrangement, i.e., the pattern, which creates depth/height illusions. Similarly, changing size and spacing of repeated closed figures, such as an oval, produces a desired arrangement that also creates elevation based illusions. Particular types of suitable illusions, alone or in combination, include geometric, spiral, anomalous motion, rotational, color change, peripheral drift, positive after image blurs, scintillation grid, stereopsis and chromostreopsis, contraction and expansion, contrast polarities, convection, and chromatic assimilation. Additionally, the optical illusions of motion may further implement optical illusionary elevation deviation relative to an exterior surface of the substrate 102 either in separate illusions or one integral illusion.

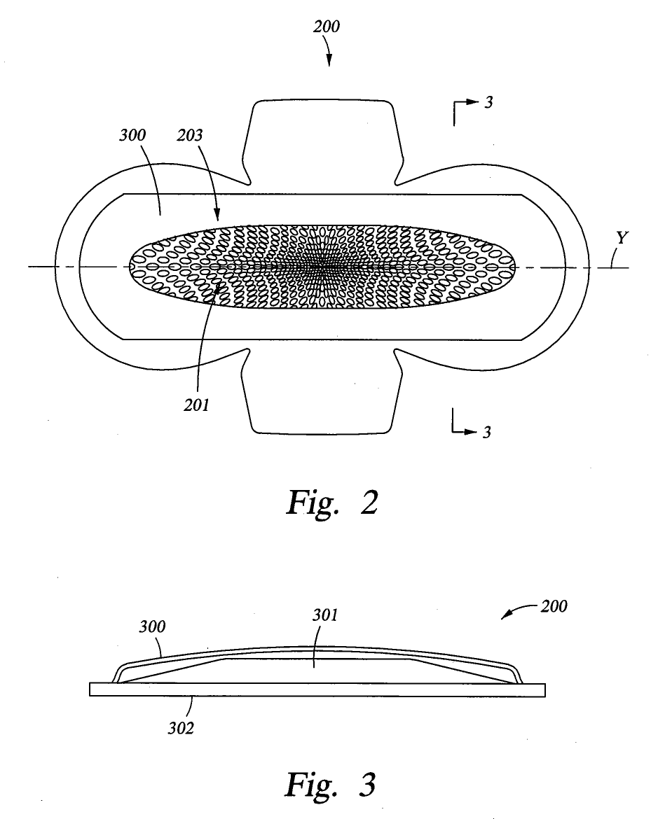

FIG. 2 illustrates an application in which a feminine hygiene pad 200 utilizes an optical illusion of motion and elevation deviation formed by a plurality of graphical elements 201 arranged in a pattern 203. The graphical elements 201 include oval shapes defined by space between the ovals being a different color tone (e.g., blue) than the ovals, which may be the color (e.g., white) of the pad 200 itself. The oval shapes making up the graphical elements 201 each include a major axis oriented to intersect the center of the pad 200. In the illustrative embodiment, the pattern 203 itself has an elliptical shape with the ovals being symmetrically disposed relative to a major axis Y of the pattern 203. Specifically, a substantial number of the ovals on either side of the center of the pattern 203 form a plurality of V-shaped arrangements, having their respective vertices disposed on the major axis of the pattern 203. The respective segments of each V-shaped arrangement traverse a divergent arcuate path, thus creating the appearance of a channel formed along the major axis of the pattern 203. Further, the density of the oval shapes increases with proximity to the center of the pad 200. In the illustrative embodiment, the increasing density toward the center of the pad is achieved by the provision of increasingly smaller oval shapes and increasingly less separation between adjacent oval shapes. In this arrangement, the graphical elements 201 appear to move to a center of the pad 200 creating the illusion of depth. Whether or not a presence or absence of coloring of the pad 200 provides the graphical elements 201, only a single color tone, single hue, and/or single brightness is necessary to provide the pattern for the illusion, which may be enhanced by multiple color tones if desired to augment the illusion created by the pattern itself. Use of a single color tone may reduce process complexity and cost associated with applications having multiple color tones.

The illusion provided by the graphical elements 201 creates a perception of movement to its center along with thickness and depth that simulates fluid flowing and moving through or deeper into the pad 200. These perceptions correlate to enhancements in protection and comfort to the user. Pads offering greater protection may utilize an illusion that appears to have more movement and/or greater elevation deviation relative to an illusion on pads that provide less protection. Patterns that provide graphical illusions may also, or alternatively, be disposed on packaging containing the pad 200 and that is removed prior to use. In one embodiment, a pattern on the packaging may be the same as the pattern formed on the product (e.g., the pad 200), thereby reinforcing the illusion visible on the pad 200. Alternatively, the packaging may include a different illusionary pattern of graphical elements to provide a further cue to the user, thereby facilitating appropriate selection.

FIG. 3 shows a cross-section of the pad 200 taken at line 3-3 of FIG. 2. In the illustrated embodiment, the pad 200 includes a liquid pervious topsheet 300, a substantially liquid impervious backsheet 302 joined to the topsheet 300, and an absorbent core 301 positioned and held between the topsheet 300 and the backsheet 302. As shown, the liquid pervious topsheet 300 and the liquid impervious backsheet 302 provide exemplary exterior surfaces of the pad 200. The pad 200 may also include other components, such as liquid wicking layers, liquid intake layers, liquid distribution layers, transfer layers, and barrier layers, as well as combinations thereof, which all may be highlighted by the graphical elements 201. For example, an outer boundary of the pattern 203 may correspond to starting of the barrier layers. Further, illusions of depth, such as provided by the pattern 203, may indicate presence of the liquid intake layers and/or transfer layers. In some embodiments, the movement associated with the graphical elements 201 may identify features such as the liquid distribution layers and/or transfer layers. With the topsheet 300 and backsheet 302, the pad 200 respectively provides a body-facing surface and a garment-facing surface.

In some embodiments, the topsheet 300 includes a woven fabric, a nonwoven fabric, a polymer film, a film-fabric laminate, as well as combinations thereof. Examples of a nonwoven fabric include spunbond fabric, meltblown fabric, coform fabric, a carded web, a bonded-carded-web, a bicomponent spunbond fabric as well as combinations thereof. Other examples of suitable materials for constructing the topsheet 300 include rayon, bonded carded webs of polyester, polypropylene, polyethylene, nylon, or other heat-bondable fibers, polyolefins, such as copolymers of polypropylene and polyethylene, linear low-density polyethylene, aliphatic esters such as polylactic acid, finely perforated film webs, net materials, as well as combinations thereof. A more particular example of a suitable material for the topsheet 300 includes a bonded-carded-web composed of polypropylene and polyethylene. Other examples of suitable materials include composite materials of a polymer and a nonwoven fabric material. The composite materials may be in the form of integral sheets formed by the extrusion of a polymer onto a web of spunbond material. A plurality of pores, perforations, apertures or other openings, as well as combinations thereof, which are present or formed in the topsheet 300, may provide the liquid-permeability. The topsheet 300 may retain little or no liquid in its structure and be configured to provide a comfortable and non-irritating surface next to the body of the user.

For some embodiments, the backsheet 302 includes a polymeric film, a woven fabric, a nonwoven fabric, as well as combinations or composites thereof. For example, the backsheet 302 may include a polymer film laminated to a woven or nonwoven fabric. The polymer film may be composed of polyethylene, polypropylene, polyester, as well as combinations thereof. The backsheet 302 may permit passage of air and moisture vapor out of the pad 200 while blocking the passage of bodily liquids. An example of a suitable material includes a breathable, microporous film. The material is a breathable film, which is dimple embossed and contains: 47.78% calcium carbonate, 2.22% TiO₂, and 50% polyethylene. The polymer film may have a minimum thickness of about 0.025 millimeters (mm) and a maximum thickness of about 0.13 mm. Bicomponent films or other multi-component films may also be used, as well as woven and/or nonwoven fabrics which have been treated to render them liquid-impermeable. Another suitable material for the backsheet 302 includes closed cell polyolefin foam, such as closed cell polyethylene foam.

Structure of the absorbent core 301 provides desired levels of liquid retention and storage capacity and desired levels of liquid acquisition and distribution. The absorbent core 301 holds a liquid, such as urine, menses, other complex biological liquid, as well as combinations thereof. The absorbent core 301 may include a matrix of absorbent fibers and/or absorbent particulate material, and the absorbent fiber may include natural and/or synthetic fiber. The absorbent core 301 may also include one or more components that modify menses or inter-menstrual liquids. The absorbent core 301 may also include superabsorbent material, such as a water-swellable, generally water-insoluble, hydrogel-forming polymeric absorbent material, which is capable of absorbing at least about 20, about 30, or about 60 times or more its weight in physiological saline (e.g., 0.9 wt % NaCl). The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, and polyvinyl pyridine. Other suitable hydrogel-forming polymers include hydrolyzed acrylonitrile grafted starch, acrylic acid grafted starch, and isobutylene maleic anhydride copolymers and mixtures thereof. The hydrogel-forming polymers may be lightly crosslinked to render the material substantially water insoluble. Crosslinking may occur by irradiation or covalent, ionic, Van der Waals, or hydrogen bonding.

In one embodiment, printing an ink onto the topsheet 300 or a layer visible under the topsheet 300 (i.e., the topsheet 300 may be transparent or translucent) provides one method of integrating the graphical elements 201 into the pad 200 or any other substrate. The graphical elements 201 may thus be patterned on an internal part of the pad 200 such that they are protected from rubbing off while still being visible through to the exterior surface. For some embodiments, an ink jet printer applies the graphical elements 201. While directly printed graphics are economical and time efficient, embossing and/or slitting techniques also enable making the graphical elements 201 visible. A combination of these techniques is also contemplated.

In some embodiments, the graphical elements are initially at least partially invisible or lighter and become visible or darker when exposed to some stimulus or substance (e.g., urine). Further, the graphical elements may be initially visible as a first color and become invisible, lighter or a second color when exposed to the stimulus or substance. For example, suitable compositions can include an ink that creates the pattern 203 and changes color when exposed to an aqueous solution such as urine.

Such active graphics can utilize a fading graphic or an appearing graphic, whereupon a color change composition can be adapted to blend in with a background or surrounding color, either before or after exposure to the aqueous solution. The active graphic can include pH sensitive inks, fugitive inks, colored absorbent particles, hydratable salts, moisture sensitive films, enzymes, and/or heat sensitive inks and dyes. For some embodiments, suitable compositions of this color-change type may be based on color changes of pH indicating molecules, formation or degradation of colored complexes, the action of one or more enzymes that occur naturally in urine on colored molecules, oxidation and/or reduction reactions, and reactions of two or more components that are spatially separated within a product, at least one of which is soluble in urine, and which upon contact, react to create or destroy a colored region. Further examples that achieve color changes include the pre-combination of two colors to form a blended color, after the wetting of which one color component may be solubilized or destroyed to leave only the second color component visible, and molecules that exhibit different color properties in crystalline and dissolved states. Other active graphics of the absorbent article can comprise a fading graphic which is formed from a composition such as an ink that is soluble in aqueous solutions such as urine. Once dissolved, the composition washes away from an outer cover and is obscured by the outer cover making the active graphic seem to disappear from view.

In one embodiment, the relative hydration of an absorbent product may be indicated by the increasing “depth” of a graphical pattern as the level of liquid absorbed increases. Maximum hydration may be related to a maximum “depth” of the graphical pattern, where the maximum “depth” can be indicated by a change in color of the pattern. With reference to the pad 200 shown in FIG. 2, for example, hydration may initially cause the perimeter of the pattern 203 to become visible. With increasing hydration, the pattern 203 may become more visible, moving from the perimeter to the center. Maximum hydration is reached when the center of the pattern is visible.

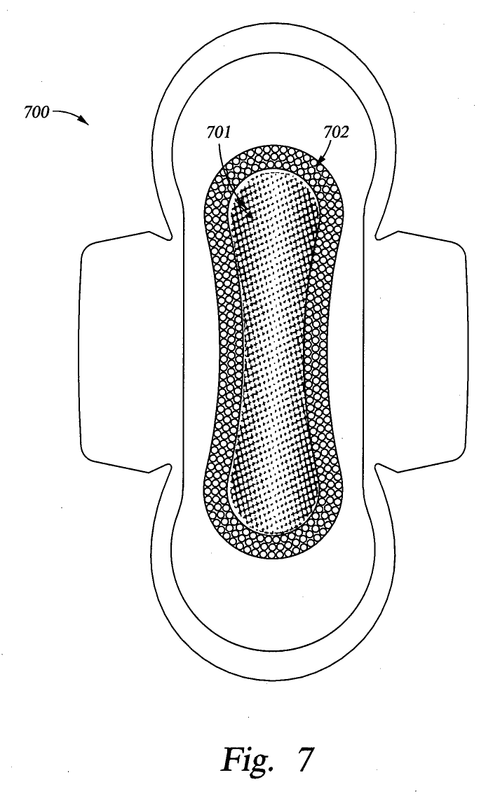

FIGS. 4-6 illustrate exemplary optical illusions viewable on external surfaces of substrates. In FIG. 4, oval graphical elements 401 oriented in a common direction and aligned in columns and rows form a pattern creating an optical illusion that appears to move down and into the substrate along two vertical (relative to a depicted orientation) regions 402. The vertical regions 402 are defined in areas where the oval graphical elements 401 become skinnier and closer together relative to adjacent areas 404, 405, 406 on either side of the regions 402 where the oval graphical elements 401 are wider and less dense. Similar to the pad 200, the oval graphical elements 401 may simulate movement of fluid into the substrate at the vertical regions 402. The user receives a visual cue that the fluid introduced along a central area 405 flows to the vertical regions 402 without escaping vertical side edges of the substrate since any of the fluid at first and second side areas 404, 406 on each side of the central area 405 also according to the visual cue flows to the vertical regions 402. When implemented, for example, on a pad or diaper, the illusion makes the user understand that urine applied to the central area 405 is distributed toward both the side areas 404, 406 and drawn in for maximum efficiency and comfort without escaping from the side areas 404, 406 and thereby leaking.

As shown in FIG. 5, a pattern utilizes a plurality of rectangular graphical elements 501 to create an optical illusion that appears to move down and into the substrate along a vertical region 502. The rectangular graphical elements 501 alternate in a checker board manner and are each sized progressively skinnier and closer together toward the vertical region 502. The user viewing the substrate perceives as a visual cue movement converging toward the vertical region 502 that appears deeper into the substrate than an actual contour profile of the substrate at the vertical region 502. Similar to the pad 200 shown in FIG. 2, the illusion may be incorporated on a pad or diaper to convey to the user that urine is quickly drawn in along the vertical region 502.

FIG. 6 illustrates an optical illusionary elevation deviation that includes both depth into the substrate and height out of the substrate. First and second patterned half-ball shaped objects 604, 606 appear to come out of the plane of the substrate while a central background pattern 602 appears deeper into the plane of the substrate. The central background pattern 602 includes uniform sized circular shaped graphical objects aligned in rows and columns. The ball shaped objects 604, 606 also have round shaped graphical objects that are ordered next to one another and become smaller and more elliptical toward an interface with the background pattern 602. At least some of the round shaped graphical objects of the ball shaped objects 604, 606 are larger than the circular shaped graphical objects of the background pattern 602. By way of example, the ball shaped objects 604, 606 may correspond to internal barrier layers within a pad while the central background pattern 602 identifies where internal absorbent media is within the pad. Providing this information to a user may allow the user to properly position the pad relative to the user's body.

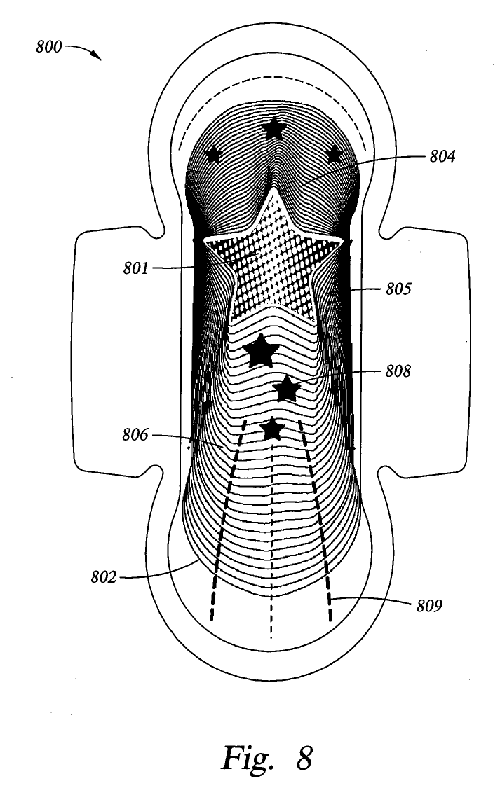

FIG. 7 shows one embodiment of a feminine hygiene pad 700 utilizing optical illusions in which graphical elements are shaped and arranged to define a first pattern 701 and a second pattern 702. The second pattern 702 surrounds the first pattern 701 and is different from the first pattern 701. The first pattern 701 includes an illusion of depth. For example, a plurality of oval shapes oriented in one direction and aligned in diagonal rows relative to a major axis of the oval shapes form the first pattern 701 with the shapes becoming increasing closer toward a longitudinal center line of the pad 700. This arrangement is more clearly seen in the patterned star shape 801 illustrated in FIG. 8. While schematically depicted as overlapping such that distinct oval shapes are not apparent at the longitudinal center line, the first pattern 701 may have spacing between the oval shapes that converges while maintaining distinct delineation among adjacent oval shapes. The oval shapes of the first pattern 701 are lighter than interspacing regions between the oval shapes such that, as the oval shapes become more densely arranged, the first pattern 701 appears to become lighter at the longitudinal center line. This convergence of spacing between the oval shapes and change in lightness makes the user perceive that the pad 700 has added depth where the first pattern 701 is located. In the illustrated embodiment, the second pattern 702 includes geometric shapes, illustratively circles. When disposed adjacent the first pattern 701, the circular shapes create an illusion of height out of a surface of the pad 700 due to the second pattern 702 delineating in a bounded formation the perceived depth at the first pattern 701 such that a remainder of the pad 700 is thus perceived as an intermediate depth reference point. Thus, the second pattern 702 provides a perception of a raised boundary for perceived fluid holding created by the first pattern 701, thereby visually indicating that the fluid is maintained in the pad 700 without escaping.

FIG. 8 illustrates another embodiment of a feminine hygiene pad 800 utilizing optical illusions of depth in which graphical elements include a five point patterned star shape 801 and lines 802. The graphical elements focus attention to the patterned star shape 801, which is patterned with oval shapes to create an illusion of height relative to the lines 802 that appear to have a lower depth on the pad 800. As shown, the patterned star shape 801 includes an arrangement like the first pattern 701 depicted in FIG. 7. Identifying to the user a location of relative increased absorbency with the patterned star shape 801 ensures that the user wears the pad 800 in a proper orientation.

The lines 802 surround the patterned star shape 801 and are spaced from one another to provide an illusion of topographical variances relative to a top surface of the pad 800. The lines 802 transition from an outline of the patterned star shape 801 in close proximity to the patterned star shape 801 to a general shape of the pad 800. A first portion 804 of the lines 802 that are more closely spaced and curved appear to have a cup shape ahead of the star shape 801 where the lines 802 tend to round out away from a V-shape at a top of the patterned star shape 801. At sides of the pad 800, a second portion 805 of the lines 802 get progressively closer together at edges of the pad 800 to provide an illusion of receding toward the edges for a perceived more comfortable fit of the pad 800. The lines 802 at the second portion 805 progressively flatten out from concavities on each side of the patterned star shape 801. A third portion 806 of the lines 802 that are farther apart and that trail the star shape 801 appear relatively more raised to suggest conformance to the body of the user. The lines 802 at the third portion 806 progressively invert to a convex curve relative to a concavity of the patterned star shape 801.

Further, the third portion 806 of the lines 802 appears to define a channel through which star shaped graphics 808 seem to be moving. The channel is formed by an interface between the second and third portions 805, 806 of the lines 802. Dashed lines 809 extending away from the star shaped graphics 808 enhance impressions that the star shaped graphics 808 are moving toward the patterned star shape 801. Thus, the star shaped graphics 808 appear to be shooting with tails provided by the dashed lines 809.

As apparent from the foregoing description, various issues, such as toddlers on the go, odor control, controlled fit, and fluid management (channeling or cleaning), correspond to features and benefits of products that are designed to address the issues. For example, the corresponding features may allow the toddlers to move more freely, include additives to control odor, provide materials that allows for better movement in areas such as waistband and leg openings, channel fluid, and/or remove bodily fluid from a body of a user. Graphics as discussed herein can respectively highlight enhanced movement of a diaper and thereby appear to allow the toddler to move more freely, appear to move or react to absorb odor, highlight enhanced flexibility of the product such as by appearing to stretch better with the body, and/or appear to move fluid to a different area of the product or off the body by wiping or cleaning the body.

It should be understood that the various shapes and colors disclosed herein are merely illustrative. Persons skilled in the art, having the benefit of the present disclosure, will appreciate that other shapes and colors may be used. While the foregoing is directed to embodiments of the present invention, other and further embodiments of the invention may be devised without departing from the basic scope thereof, and the scope thereof is determined by the claims that follow.

What is claimed is:

1. An absorbent personal care article, comprising:
a body having internal absorbent media and an exterior surface; and
a plurality of graphical elements visible along the exterior surface, wherein the graphical elements form a pattern that creates an optical illusion of motion.

2. The article of claim 1, wherein actual appearance of the graphical elements changes in response to a stimulus applied to the body by a user of the article.

3. The article of claim 1, wherein the exterior surface comprises an outer cover opposite a user facing surface of the body such that the graphical elements appear as moving objects on an outside of the article.

4. The article of claim 1, wherein the graphical elements are patterned on an internal part of the body visible through a layer defining the exterior surface.

5. The article of claim 1, wherein the graphical elements are at a region of the body containing a visibly indiscernible component that provides at least one functional aspect associated with use of the article.

6. The article of claim 1, wherein the graphical elements are arranged relative to one another to form the optical illusion of motion perceivable by at least one of the elements appearing to move away from an edge of the body.

7. The article of claim 1, wherein at least some of the plurality of graphical elements are arranged to create an optical illusionary elevation deviation relative to the exterior surface.

8. The article of claim 1, wherein at least some of the plurality of graphical elements are arranged to create an optical illusionary elevation deviation into the exterior surface in a central region of the body and out of the exterior surface in an area bordering the central region.

9. The article of claim 1, wherein all the graphical elements have a single common color tone.

10. The article of claim 1, further comprising a first liquid pervious cover layer and a second liquid impervious layer, wherein the absorbent media is disposed between the layers.

11. A personal care article, comprising:
a body having a region containing a visually indiscernible component of the article that provides at least one functional aspect associated with use of the article; and
a plurality of graphical elements visible on the body at the region, wherein the graphical elements are static and arranged relative to one another such that a viewer perceives at least one of the elements with a first characteristic different than a second characteristic later in time.

12. The article of claim 11, wherein the first and second characteristics are perceivable by the viewer dependent on a focal point of the viewer relative to the plurality of graphical elements.

13. The article of claim 11, wherein the body is disposable.

14. The article of claim 11, wherein the component comprises an absorbent media.

15. The article of claim 11, wherein the component comprises a substance upon which activation depends on a stimulus applied to the region by a user of the article.

16. The article of claim 11, wherein the component comprises an odor control substance activated by moisture.

17. The article of claim 11, wherein the component comprises an active therapeutic substance.

18. The article of claim 11, wherein the component is invisible to the naked eye of the viewer.

19. An absorbent personal care article, comprising:
a body having internal absorbent media between a liquid pervious cover layer and a liquid impervious layer; and
a plurality of graphical elements visible on an exterior surface of the body from a side of the body having the liquid pervious cover layer, wherein the graphical elements are arranged relative to one another to form an optical illusion of motion perceivable by at least one of the elements appearing to move away from an edge of the body.

20. The article of claim 19, further comprising removable packaging in which the body is disposed prior to use, wherein a design on the packaging produces the optical illusion of motion.

21. The article of claim 19, wherein the graphical elements comprise closed shapes of different sizes separated by different distances from one another.

22. The article of claim 19, wherein the graphical elements comprise lines with at least one of thicknesses and spacing of the lines being varied.

23. The article of claim 19, wherein the graphical elements are shaped and arranged to define a first pattern that forms the optical illusion of motion and a second pattern different from the first pattern.

24. The article of claim 19, wherein the graphical elements are shaped and arranged to define a first pattern and a second pattern that surrounds the first pattern and is different from the first pattern.

25. The article of claim 19, wherein the graphical elements are printed onto the liquid pervious cover layer.
